(19) United States
(12) Patent Application Publication
(10) Pub. No.: US 2024/0042161 A1
(43) Pub. Date: Feb. 8, 2024

(54) System and Method for Operating a Ventilator

(71) Applicants: Carnegie Mellon University, Pittsburgh, PA (US); University of Pittsburgh - Of the Commonwealth System of Higher Education, Pittsburgh, PA (US)

(72) Inventors: Lu Li, Pittsburgh, PA (US); Michael B. Schwerin, Pittsburgh, PA (US); Howie Choset, Pittsburgh, PA (US); Keith E. Cook, Pittsburgh, PA (US); Jason Rose, Blawnox, PA (US)

(21) Appl. No.: 18/245,936

(22) PCT Filed: Sep. 17, 2021
(86) PCT No.: PCT/US21/50899

§ 371 Date: Mar. 20, 2023

Related U.S. Application Data
(60) Provisional application No. 63/080,387, filed on Sep. 18, 2020.

(51) Int. Cl. A61M 16/20; A61M 16/10; G06N 20/00

(52) U.S. Cl. CPC A61M 16/204; A61M 16/1005; A61M 16/205; G06N 20/00; A61M 2205/3372; A61M 2016/0027

(57) ABSTRACT

Provided is a system for operating a ventilator. The system includes a motorized proportional valve actuator including a stepper motor and an actuator. The actuator is connected to the stepper motor and configured to output pressurized air by controlling a pressure on a valve diaphragm. A conduit provides for fluid communication of the pressurized air to a breathing apparatus. A sensor arrangement is in fluid communication with the conduit between the at least one motorized proportional valve actuator and the breathing apparatus. The sensor arrangement includes: (i) an intake manifold configured to output a restricted flow of air from the pressurized air transported in the conduit, and (ii) a sensor device in fluid communication with an outlet of the intake manifold, the sensor device configured to measure an air pressure of the conduit based on the restricted flow of air.

### CROSS REFERENCE TO RELATED APPLICATION

This application claims priority to U.S. Provisional Patent Application No. 63/080,387, filed Sep. 18, 2020, the disclosure of which is hereby incorporated by reference in its entirety.

### GOVERNMENT LICENSE RIGHTS

This invention was made with United States government support under HL136857 awarded by the National Institutes of Health. The U.S. government has certain rights in the invention.

### BACKGROUND OF THE INVENTION

### 1. Field

This disclosure relates generally to ventilators and, in non-limiting embodiments, to a system and method for operating a ventilator with increased reliability and quicker manufacturing.

### 2. Technical Considerations

Ventilators are often used in emergency situations to help patients who are unable to breath on their own. They often require very specialized equipment which can be difficult to procure quickly. In situations in which a large number of people need access to ventilators in a short period, as experienced during the COVID-19 pandemic, the delivery of additional ventilators may be unable to meet demands due to certain components used in manufacturing ventilators. Further, the components of ventilators often take a significant amount of time to repair due to the difficulties in obtaining replacement parts. Additionally, a significant amount of medical resources, such as the time of doctors and nurses, is often spent monitoring and adjusting ventilators in the rooms in which they are located. This results in significant exposures to medical personnel to potentially dangerous contagions from infected patients and also decreases the availability of medical personnel to respond to emergencies. Therefore, there is a need to provide a ventilator with an improved design to allow for increased availability and manufacturing of the ventilators as well as improve the operations to increase the availability of healthcare workers.

### SUMMARY

According to non-limiting embodiments or aspects, provided is a ventilator including at least one motorized proportional valve actuator, each motorized proportional valve actuator comprising a stepper motor and an actuator, the actuator connected to the stepper motor and configured to output pressurized air by controlling a pressure on a valve diaphragm. A conduit may provide for fluid communication of the pressurized air to a breathing apparatus. A sensor arrangement may be in fluid communication with the conduit between the at least one motorized proportional valve actuator and the breathing apparatus, the sensor arrangement may include: (i) an intake manifold configured to output a restricted flow of air from the pressurized air transported in the conduit, and (ii) a sensor device in fluid communication with an outlet of the intake manifold, the sensor device configured to measure an air pressure of the conduit based on the restricted flow of air.

In non-limiting embodiments or aspects, the ventilator may comprise a processor in communication with the sensor device, the processor may be configured to determine a temperature compensation and a humidity compensation. The sensor device may be configured to measure a pressure range of −200 cm H₂O to 200 cm H₂O and a flow rate range of 0 to 100 L/min. The motorized proportional valve actuator may include a plurality of motorized proportional valve actuators including an oxygen proportional valve configured to adjust oxygen concentration levels, an inspiratory proportional valve configured to adjust air pressure of inspiratory air, and an expiratory proportional valve configured to adjust air pressure of expiratory air. The inspiratory proportional valve actuator may be configured to operate in an inspiratory range of 0-120 cm H₂O and the expiratory pressure motorized proportional valve actuator may be configured to operate in an expiratory range of 0-25 cm H₂O. The intake manifold may include a top plate and a bottom plate, wherein connecting the top plate to the bottom plate forms an inspiratory conduit and an expiratory conduit, the inspiratory conduit being independent of the expiratory conduit.

According to non-limiting embodiments or aspects, provided is a central controller for a ventilator including one or more processors programmed and/or configured to receive an input signal from a sensor arrangement in fluid communication with a conduit between at least one motorized proportional valve actuator and a breathing apparatus providing for fluid communication of pressurized air to the breathing apparatus, the sensor arrangement may include (i) an intake manifold configured to output a restricted flow of air from the pressurized air transported in the conduit, and (ii) a sensor device in fluid communication with an outlet of an intake manifold, the sensor device configured to measure an air pressure of the conduit based on the restricted flow of air. The one or more processors may be programmed and/or configured to determine an actuator movement based on the input signal; and communicate an instruction based on the actuator movement to a motorized proportional valve actuator comprising a stepper motor and an actuator, the actuator connected to the stepper motor and configured to output the pressurized air by controlling a pressure on a valve diaphragm, wherein the instruction directs the stepper motor to complete the actuator movement to change the pressure on the valve diaphragm.

In non-limiting embodiments or aspects, the central controller may be wireless and arranged remote from the ventilator, and configured to be in wireless communication with at least one motorized proportional valve actuator of at least two ventilators. The central controller may be in communication with a user device, wherein the central controller operates the at least one motorized proportional valve actuator based on an input signal received from the user device. Determining an actuator movement based on the input signal may be determined based on a machine learning algorithm.

According to non-limiting embodiments or aspects, provided is a method of operating a ventilator including receiving, with at least one processor, an input signal from at least one sensor arrangement in fluid communication with a conduit, the conduit establishing fluid communication between at least one motorized proportional valve actuator and a breathing apparatus, the sensor arrangement may include: (i) an intake manifold configured to output a restricted flow of air from pressurized air transported in the conduit, and (ii) at least one sensor device in fluid communication with an outlet of the intake manifold, the sensor device configured to measure an air pressure of the conduit based on the restricted flow of air. The method may include adjusting, with at least one processor, a stepper motor of a motorized proportional valve actuator based on comparing the input signal to a predetermined value, wherein the motorized proportional valve actuator comprises an actuator connected to the stepper motor, the actuator configured to output pressurized air by controlling a pressure on a valve diaphragm.

In non-limiting embodiments or aspects, the method may further include determining, with at least one processor, a temperature compensation and a humidity compensation of the at least one sensor device. The sensor device may be configured to measure a pressure range of −200 cm H₂O to 200 cm H₂O and a flow rate range of 0 to 100 L/min. The motorized proportional valve actuator may include a plurality of motorized proportional valve actuators including an oxygen proportional valve configured to adjust oxygen concentration levels, an inspiratory proportional valve configured to adjust air pressure of inspiratory air, and an expiratory proportional valve configured to adjust air pressure of expiratory air. The inspiratory pressure motorized proportional valve actuator may be configured to operate in an inspiratory range of 0-120 cm H₂O and the expiratory pressure motorized proportional valve actuator is configured to operate in an expiratory range of 0-25 cm H₂O. The intake manifold may include a top plate and a bottom plate, wherein connecting the top plate to the bottom plate forms an inspiratory conduit and an expiratory conduit, the inspiratory conduit being independent of the expiratory conduit.

Other non-limiting embodiments or aspects will be set forth in the following numbered clauses:

Clause 1: A ventilator comprising: at least one motorized proportional valve actuator, each motorized proportional valve actuator comprising a stepper motor and an actuator, the actuator connected to the stepper motor and configured to output pressurized air by controlling a pressure on a valve diaphragm; a conduit providing for fluid communication of the pressurized air to a breathing apparatus; and a sensor arrangement in fluid communication with the conduit between the at least one motorized proportional valve actuator and the breathing apparatus, the sensor arrangement comprising: (i) an intake manifold configured to output a restricted flow of air from the pressurized air transported in the conduit, and (ii) a sensor device in fluid communication with an outlet of the intake manifold, the sensor device configured to measure an air pressure of the conduit based on the restricted flow of air.

Clause 2: The ventilator of clause 1, further comprising a processor in communication with the sensor device, the processor configured to determine a temperature compensation and a humidity compensation.

Clause 3: The ventilator of clauses 1 or 2, wherein the sensor device is configured to measure a pressure range of −200 cm H₂O to 200 cm H₂O and a flow rate range of 0 to 100 L/min.

Clause 4: The ventilator of any of clauses 1-3, wherein the motorized proportional valve actuator comprises a plurality of motorized proportional valve actuators including an oxygen proportional valve configured to adjust oxygen concentration levels, an air inspiratory proportional valve configured to adjust air pressure of inspiratory air, and an expiratory proportional valve configured to adjust air pressure of expiratory air.

Clause 5: The ventilator of any of clauses 1-4, wherein the inspiratory proportional valve actuator is configured to operate in an inspiratory range of 0-120 cm H₂O and the expiratory pressure motorized proportional valve actuator is configured to operate in an expiratory range of 0-25 cm H₂O.

Clause 6: The ventilator of any of clauses 1-5, wherein the intake manifold comprises a top plate and a bottom plate, wherein connecting the top plate to the bottom plate forms an inspiratory conduit and an expiratory conduit, the inspiratory conduit being independent of the expiratory conduit.

Clause 7: A central controller for a ventilator comprising: one or more processors programmed and/or configured to: receive an input signal from a sensor arrangement in fluid communication with a conduit between at least one motorized proportional valve actuator and a breathing apparatus providing for fluid communication of pressurized air to the breathing apparatus, the sensor arrangement comprising: (i) an intake manifold configured to output a restricted flow of air from the pressurized air transported in the conduit, and (ii) a sensor device in fluid communication with an outlet of an intake manifold, the sensor device configured to measure an air pressure of the conduit based on the restricted flow of air; determine an actuator movement based on the input signal; and communicate an instruction based on the actuator movement to a motorized proportional valve actuator comprising a stepper motor and an actuator, the actuator connected to the stepper motor and configured to output the pressurized air by controlling a pressure on a valve diaphragm, wherein the instruction directs the stepper motor to complete the actuator movement to change the pressure on the valve diaphragm.

Clause 8: The central controller of clause 7, wherein the central controller is wireless and arranged remote from the ventilator, and configured to be in wireless communication with at least one motorized proportional valve actuator of at least two ventilators.

Clause 9: The central controller of clauses 7 or 8, wherein the central controller is in communication with a user device, wherein the central controller operates the at least one motorized proportional valve actuator based on an input signal received from the user device.

Clause 10: The central controller of any of clauses 7-9, wherein determining an actuator movement based on the input signal is determined based on a machine learning algorithm.

Clause 11: A method of operating a ventilator, comprising: receiving, with at least one processor, an input signal from at least one sensor arrangement in fluid communication with a conduit, the conduit establishing fluid communication between at least one motorized proportional valve actuator and a breathing apparatus, the sensor arrangement comprising: (i) an intake manifold configured to output a restricted flow of air from pressurized air transported in the conduit, and (ii) at least one sensor device in fluid communication with an outlet of the intake manifold, the sensor device configured to measure an air pressure of the conduit based on the restricted flow of air; and adjusting, with at least one processor, a stepper motor of a motorized proportional valve actuator based on comparing the input signal to a predetermined value, wherein the motorized proportional valve actuator comprises an actuator connected to the stepper motor, the actuator configured to output pressurized air by controlling a pressure on a valve diaphragm.

Clause 12: The method of clause 11, further comprising determining, with at least one processor, a temperature compensation and a humidity compensation of the at least one sensor device.

Clause 13: The method of clauses 11 or 12, wherein the sensor device is configured to measure a pressure range of −200 cm H₂O to 200 cm H₂O and a flow rate range of 0 to 100 L/min.

Clause 14: The method of any of clauses 11-13, wherein the motorized proportional valve actuator comprises a plurality of motorized proportional valve actuators including an oxygen proportional valve configured to adjust oxygen concentration levels, an inspiratory proportional valve configured to adjust air pressure of inspiratory air, and an expiratory proportional valve configured to adjust air pressure of expiratory air.

Clause 15: The method of any of clauses 11-14, wherein the inspiratory pressure motorized proportional valve actuator is configured to operate in an inspiratory range of 0-120 cm H₂O and the expiratory pressure motorized proportional valve actuator is configured to operate in an expiratory range of 0-25 cm H₂O.

Clause 16: The method of any of clauses 11-15, wherein the intake manifold comprises a top plate and a bottom plate, wherein connecting the top plate to the bottom plate forms an inspiratory conduit and an expiratory conduit, the inspiratory conduit being independent of the expiratory conduit.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Additional advantages and details are explained in greater detail below with reference to the exemplary embodiments that are illustrated in the accompanying schematic figures, in which:

FIG. 1 is a schematic diagram of a system for operating a ventilator according to non-limiting embodiments or aspects;

FIG. 2 is a diagram of a motorized proportional valve actuator according to non-limiting embodiments or aspects;

FIGS. 3A-3C are diagrams of a manifold for a ventilator according to non-limiting embodiments or aspects;

FIG. 4 is a flow chart of a method of operating a ventilator according to non-limiting embodiments or aspects;

FIG. 5 is a schematic diagram of a control system for at least two ventilators according to non-limiting embodiments or aspects; and

FIG. 6 is a schematic diagram of a computing device.

### DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “computing device” and “user device” may refer to one or more electronic devices configured to process data. A computing device or user device may, in some examples, include the necessary components to receive, process, and output data, such as a processor, a display, a memory, an input device, a network interface, and/or the like. For example, a user device may include one or more computers, portable computers, laptop computers, tablet computers, mobile devices, cellular phones, wearable devices (e.g., watches, glasses, lenses, clothing, and/or the like), and/or the like. The term “user,” as used herein, may refer to an entity (e.g., a medical practitioner, doctor, nurse, and/or the like) that owns, utilizes, and/or operates a user device.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

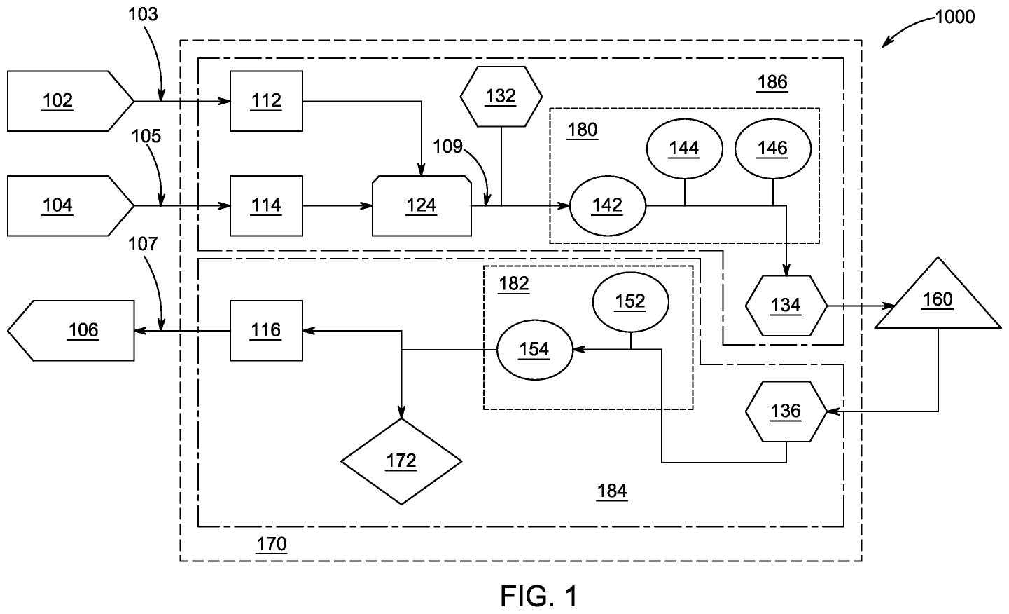

FIG. 1 depicts a system diagram for a ventilator system 1000 according to some non-limiting embodiments or aspects. The ventilator system 1000 includes an oxygen source 102, an air source 104, an air sink 106, a breathing apparatus 160, and a manifold 170. The breathing apparatus 160 may include a device (e.g., a mask and/or tube) for placement on a patient's head and/or face that allows air to pass through the device into the lungs of the patient while also receiving air exhaled from the patient and allowing the exhaled air to pass out through the device. The ventilator system 1000 may be modularly designed such that individual components may be repaired or replaced without impacting other components of the ventilator system 1000. In non-limiting embodiments, the ventilator system 1000 can be manufactured at a low-cost, can be mass produced in large quantities, and may be controlled remotely with reliable performance. The modular design allows for quick repairs to minimize downtime due to a break or malfunction of a component. The manifold 170 may enclose and protect embedded sensors and processors. Remote operation of the ventilator system allows for caregivers to avoid potential exposure to infections by allowing ventilator operations to be controlled from outside of the room housing the patient, which will also result in a decreased need for the use of personal protective equipment. The manifold 170 may be configured to be removed and replaced for easy sterilization between uses. Full assembly of each ventilator system 1000 may occur in less time than typical ventilators, and in some examples may be achieved in less than one hour per machine.

With continued reference to FIG. 1, the oxygen source 102 provides gaseous oxygen (O₂) into the ventilator system 1000. The oxygen source 102 may be a mobile oxygen tank, a fixed oxygen tank, and/or any other source. The oxygen source 102 may transport the oxygen into the ventilator system through an oxygen conduit 103. The oxygen conduit 103 may be a rigid pipe (e.g., copper, lead, PVC, steel, etc.), a flexible pipe (e.g., rubber, plastic, flexible PVC, etc.), and/or the like. The oxygen conduit 103 may connect to an intake manifold 186. The intake manifold 186 may be part of the manifold 170 that includes both the intake manifold 186 and an exit manifold 184.

With continued reference to FIG. 1, the oxygen conduit 103 may continue to an oxygen proportional valve 112. The oxygen proportional valve 112 may be configured to adjust the oxygen concentration levels (e.g., the oxygen amount or proportion of oxygen in the air) delivered to a patient based on the amount of oxygen that is delivered through the oxygen conduit 103. The oxygen proportional valve 112 may, in some examples, be a low-cost motorized proportional valve actuator. The motorized proportional valve actuator may be made of materials and components, such as stepper motors, valve bodies, valve seats, actuators, and/or the like, that are readily available such that a supply chain shortage of the components is unlikely. The motorized proportional valve actuator may modulate air flow of a gas traveling through the motorized proportional valve actuator by fully or partially opening and closing, such as altering pressure and/or flow rate. The motorized proportional valve actuator may be modularly designed such that the valve itself can be reconfigured between a high-pressure mode to regulate inspiratory airflow and a sensitive configuration for expiratory positive end-expiratory pressure (PEEP).

In some non-limiting embodiments or aspects, the motorized proportional valve actuator may be powered by a stepper motor, such as a NEMA 17 stepper motor or any other stepper motor. Stepper motors are readily available and unlikely to see any supply chain restrictions due to a sudden high demand. The stepper motor may be attached to a valve body. The components of the valve body may, in some examples, be 3D printed or molded, and may be made of plastic, metal, and/or the like. The valve body may include a poppet connected to the stepper motor, such as by a valve stem or actuator. As the motor rotates in a first direction, the movement of the stepper motor may cause the valve body to move toward a closed position. As the motor rotates in a second direction, the movement of the stepper motor may cause the valve body to move toward an open position. The valve body may include an inlet and an outlet. As the valve body moves toward the closed position, the volumetric flow rate of the fluid traveling from the inlet to the outlet is decreased. In the fully closed position, the outlet volumetric flow rate is substantially 0. As the valve body moves toward the open position, the volumetric flow rate of the fluid traveling from the inlet to the outlet is increased. In the fully open position, the volumetric flow rate of the outlet is equal to the volumetric flow rate of the inlet.

The motorized proportional valve actuator may be modular such that some components, such as the stepper motor, valve seat, valve body, valve stem, and/or other individual components, may be replaced and/or exchanged for components with different properties. The components may be changed or altered in order to configure the motorized proportional valve actuator to operate at different differential pressures. For example, one version of the motorized proportional valve actuator may be configured for a high pressure mode (e.g., to operate in the range of 0-120 cm-H₂O differential pressure) to regulate inspiratory airflow. Another configuration of the motorized proportional valve actuator may be configured to operate in a sensitive mode (e.g., to operate in the range of 0-25 cm-H₂O differential pressure) for expiratory positive end-expiratory pressure control. The motorized proportional valve actuator may be used in numerous fields, including the medical field and robotic manufacturing and assembly.

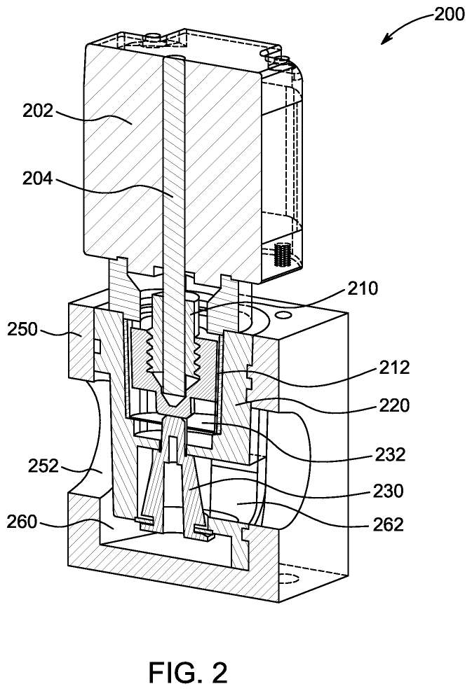

Referring now to FIG. 2, shown according to non-limiting embodiments or aspects is an example motorized proportional valve actuator 200. A motorized proportional valve actuator 200 may be powered by a stepper motor 202 with an actuator 204. The actuator 204 may be a rod attached to the stepper motor 202. As the stepper motor 202 operates, the actuator 204 is rotated. As the actuator 204 is rotated, it rotates stem spiral grooves 210 on the shaft of the actuator 204. The stem spiral grooves 210 interact with a stepper motor seat 212. As the actuator 204 rotates, the stem spiral grooves 210 cause the stepper motor seat 212 to raise or lower depending on the direction of the motor stem rotation. The stepper motor seat 212 may be enclosed in a valve body 220. The valve body 220 may be inserted into a manifold 250. A valve inlet 260 and valve outlet 262 may align with a conduit 252 of the manifold 250. As the stepper motor seat 212 lowers, it increases pressure on a valve diaphragm 232. The increased pressure on the valve diaphragm 232 pushes a valve seat 230 into an open position, allowing air to flow through the valve inlet 260 to the valve outlet 262. Decreasing pressure on the valve diaphragm 232 causes the valve seat 230 to move to a closed position, decreasing the air flow through the valve. A decreasing air flow results in a lower air pressure and an increasing air flow results in an increased air pressure.

Referring again to FIG. 1, in non-limiting embodiments or aspects, the oxygen conduit 103 may continue through the oxygen proportional valve 112 to a gas blender 124. The gas blender 124 may have two or more inlets with different gas sources being supplied to each inlet. The gas blender 124 may combine the oxygen from the oxygen source 102 with air from an air source 104. The gas blender 124 may include at least one outlet that blends the gases from each of the inlets, creating a combined gas. The oxygen concentration in the combined gas from the outlet of the gas blender 124 may be based on the position of the oxygen proportional valve 112. As the oxygen proportional valve 112 is moved to the closed position, the concentration of oxygen being supplied to the patient is decreased. As the oxygen proportional valve 112 is moved to the open position, the concentration of oxygen being supplied to the patient is increased.

With continued reference to FIG. 1, in non-limiting embodiments or aspects, the air source 104 may provide air to the manifold 170 in the ventilator system 1000. The air source 104 may be a mobile air tank, a fixed air tank, an opening to the natural air, and/or the like. The air may be transported to the manifold 170 through pressure and/or a pump. The air source 104 may transport the air into the ventilator system 1000 through an air conduit 105. The air conduit 105 may be a rigid pipe (e.g., copper, lead, PVC, steel, etc.), a flexible pipe (e.g., rubber, plastic, flexible PVC, etc.), and/or the like. The air conduit 105 may connect to the intake manifold 186. The intake manifold 186 may be part of the manifold 170.

With continued reference to FIG. 1, the air conduit 105 may continue to an inspiratory proportional valve 114. The inspiratory proportional valve 114 may be a motorized proportional valve actuator, similar to the oxygen proportional valve 112. The inspiratory proportional valve 114 may be customized to have different components than the oxygen proportional valve 112. The inspiratory proportional valve 114 may be configured to operate at the same or different pressure differentials than the oxygen proportional valve 112. The different components between the valves may include different sized and/or shaped valve seats, valve stems, valve bodies, and/or the like in order to achieve different pressure differentials. The inspiratory proportional valve 114 may be configured to regulate and adjust the air pressure of the inspiratory air that will travel through the intake manifold 186.

With continued reference to FIG. 1, in non-limiting embodiments or aspects, the air conduit 105 may continue through the inspiratory proportional valve 114 to an inlet of the gas blender 124. The gas blender 124 may blend the gases received from two or more inlets, such as from the air conduit 105 and the oxygen conduit 103.

With continued reference to FIG. 1, in non-limiting embodiments or aspects, the outlet of the gas blender 124 may be in fluid communication with an inspiratory conduit 109. The inspiratory conduit 109 provides fluid communication of air to the breathing apparatus 160. The breathing apparatus 160 may provide pressurized air to a patient for inhalation. The pressurized air may be a blended mix of natural air and oxygen. The concentration of oxygen in the air communicated through the inspiratory conduit may be between 21%-100%. In some non-limiting embodiments or aspects, the inspiratory conduit 109 may start at the inlet of the manifold 170. In some non-limiting embodiments or aspects, the inspiratory conduit 109 may be a continuation of the oxygen conduit 103 and/or the air conduit 105.

With continued reference to FIG. 1, in non-limiting embodiments or aspects, an emergency breathing check valve 132 may be in fluid communication with the inspiratory conduit 109. The emergency breathing check valve 132 may provide air to the inspiratory conduit 109 in the event that an insufficient amount of air is being provided by the oxygen source 102 or the air source 104. The inlet of the emergency breathing check valve 132 may be open to the surrounding environment and the outlet may be in fluid communication with the inspiratory conduit 109. The inlet of the emergency breathing check valve 132 may provide natural air to be communicated into the inspiratory conduit 109. The emergency breathing check valve 132 may prevent air from traveling in reverse through the emergency breathing check valve 132 (e.g., from the inspiratory conduit 109 to the environment). The emergency breathing check valve 132 may include a silicone seal that allows flow in the event of negative pressure in the inspiratory conduit 109. The silicone seal may provide a strong seal for reverse flow but minimal resistance for normal flow. In some examples, the silicone seal may be an FDA approved silicone seal.

With continued reference to FIG. 1, in non-limiting embodiments or aspects, the inspiratory conduit 109 may be in fluid communication with an inspiratory sensor arrangement 180. The inspiratory sensor arrangement 180 may include one or more sensors for measuring various properties (e.g., pressure, volumetric flow rate, velocity flow rate, humidity, temperature, air quality, etc.) of the air traveling through the inspiratory conduit 109.

With continued reference to FIG. 1, in non-limiting embodiments or aspects, one of the sensors of the inspiratory sensor arrangement 180 may be an inspiratory air flow sensor 142. The inspiratory air flow sensor 142 may measure the velocity and/or mass flow rate of the air flowing through the inspiratory conduit 109. The inspiratory air flow sensor 142 may be a rotating vane sensor, differential pressure sensor, mass airflow hot wire sensor, heat based flow sensor, rotameter, and/or the like. The differential pressure sensor may be configured to output a Venturi effect-based measurement based on an orifice ratio, for example, a ratio between 4:1 and 8:1 of the diameter of the conduit to the smallest diameter of the orifice, using two pressure sensors, one before the orifice and one after the orifice. The differential pressure sensor may be configured to include temperature and humidity compensation. The temperature and humidity compensation may be determined by a processor in communication with the differential pressure sensor. The inspiratory air flow sensor 142 may be installed in-line with the inspiratory conduit 109. The inlet of the inspiratory air flow sensor 142 may receive air flow from the gas blender 124. The outlet of the inspiratory air flow sensor 142 may direct air flow toward the breathing apparatus 160. In some non-limiting embodiments, the inspiratory air flow sensor 142 may measure flow rates up to 100 L/min. The inspiratory flow sensor 142 may be based on semiconductor components that are widely available, as opposed to conventional flow sensors which use components unique to the medical supply chain or respiratory device supply chain.

With continued reference to FIG. 1, in non-limiting embodiments or aspects, one of the sensors of the inspiratory sensor arrangement 180 may be an inspiratory pressure sensor 144. The inspiratory pressure sensor 144 may be the same or included as a component of the inspiratory air flow sensor 142 (e.g., when the inspiratory air flow sensor 142 is a differential pressure sensor that includes the inspiratory pressure sensor 144). In some non-limiting embodiments or aspects, the inspiratory pressure sensor 144 may measure pressures at ±200 cm-H₂O. In non-limiting embodiments or aspects, one of the sensors of the inspiratory sensor arrangement 180 may be an inspiratory air humidity sensor 146. The inspiratory air humidity sensor 146 may measure the humidity of the air before it reaches the breathing apparatus 160.

With continued reference to FIG. 1, in non-limiting embodiments or aspects, the outlet of the inspiratory sensor arrangement 180 may be in fluid communication with an inlet of an inspiratory check valve 134. The inspiratory check valve 134 may be configured to ensure the air flow through the inspiratory conduit 109 continues to flow out of the intake manifold 186 and does not back flow into the intake manifold 186. The inspiratory check valve 134 may be the same or similar design to the emergency breathing check valve 132. The outlet of the inspiratory check valve 134 may be in fluid communication with the breathing apparatus 160. The outlet of the inspiratory check valve 134 may direct the inspiratory conduit 109 out of the intake manifold 186 and/or the manifold 170.

With continued reference to FIG. 1, in non-limiting embodiments or aspects, the outlet of the inspiratory air conduit 109 may connect to the breathing apparatus 160. The breathing apparatus 160 may be in use by a patient such that the inspiratory air conduit 109 provides air into the lungs of a patient, either under the patient's own breathing force or through the use of mechanical assistance to aid the patient in breathing.

With continued reference to FIG. 1, in non-limiting embodiments or aspects, the outlet of the breathing apparatus 160 may be in fluid communication with an expiratory conduit 107. The expiratory conduit 107 may extend from the breathing apparatus 160 to the inlet of the exit manifold 184. The expiratory manifold 107 may transport air that has been breathed out of a patient to an air sink 106. The air sink 106 may be a tank or other container device for storing the exhaled air from the patient. The air sink 106 may also be a filtered pathway to the atmosphere. The exit manifold 184 may be a component of the manifold 170. The exit manifold 184 may have a completely separate air flow path than the intake manifold 186.

With continued reference to FIG. 1, in non-limiting embodiments or aspects, the expiratory conduit 107 may be in fluid communication with an expiratory check valve 136. The expiratory check valve 136 may be a component of the exit manifold 184. The expiratory check valve 136 may be the same or similar in design to the inspiratory check valve 134 and/or the emergency breathing check valve 132. The expiratory check valve 136 may be configured to ensure that air entering in to the manifold 170 from the breathing apparatus 160 continues to flow through the manifold and does not reverse flow back toward the breathing apparatus 160.

With continued reference to FIG. 1, the expiratory conduit 107 may provide fluid communication between the expiratory check valve 136 and an expiratory sensor arrangement 182. The expiratory sensor arrangement 182 may include one or more sensors for measuring various properties (e.g., pressure, volumetric flow rate, velocity flow rate, humidity, temperature, air quality, etc.) of the air traveling through the expiratory conduit 107.

With continued reference to FIG. 1, in non-limiting embodiments or aspects, one of the sensors of the expiratory sensor arrangement 182 may be an expiratory pressure sensor 152. In some non-limiting embodiments or aspects, the expiratory pressure sensor 152 may be configured to measure pressures at ±200 cm-H₂O. The expiratory pressure sensor 152 may the same or similar design as the inspiratory pressure sensor 144.

With continued reference to FIG. 1, in non-limiting embodiments or aspects, one of the sensors of the expiratory sensor arrangement 182 may be an expiratory air flow sensor 154. The expiratory air flow sensor 154 may be configured to measure the velocity and/or mass flow rate of the air flowing through the expiratory conduit 107. The expiratory air flow sensor 154 may be the same or a different design as the inspiratory flow sensor 142. The expiratory air flow sensor 154 may be a rotating vane sensor, differential pressure sensor, mass airflow hot wire sensor, heat based flow sensor, rotameter, and/or the like. The differential pressure sensor may be configured to provide a Venturi effect-based measurement based on an orifice ratio, for example, between 4:1 and 8:1, using two pressure sensors, one before the orifice and one after the orifice. The expiratory pressure sensor 152 may be the same or included as a component of the expiratory air flow sensor 154 (e.g., when the expiratory air flow sensor 154 is a differential pressure sensor that includes the expiratory pressure sensor 152). The expiratory air flow sensor 154 may be installed in-line with the expiratory conduit 107. The inlet of the expiratory air flow sensor 154 may receive air flow from the expiratory check valve 136. The outlet of the expiratory air flow sensor 154 may direct air flow toward the air sink 106. In some non-limiting embodiments, the expiratory air flow sensor 154 may measure flow rates between 0 L/min and 100 L/min. The expiratory flow sensor 154 may be based on semiconductor components that are widely available, as opposed to flow sensors which use components specific to the medical supply chain.

With continued reference to FIG. 1, in non-limiting embodiments or aspects, the expiratory conduit 107 may continue the fluid communication between the expiratory flow sensor 154 and an expiratory proportional valve 116. The expiratory proportional valve 116 may be configured for positive end-expiratory pressure (PEEP) adjustments (e.g., adjusting the expiratory air pressure) and may be part of the exit manifold 184 and/or the manifold 170. The expiratory proportional valve 116 may be configured to operate at pressures in the range of 0-25 cm-H₂O. The expiratory proportional valve 116 may be a motorized proportional valve actuator and may be the same or similar design as the oxygen proportional valve 112 and/or inspiratory proportional valve 114.

With continued reference to FIG. 1, in non-limiting embodiments or aspects, an emergency expiratory pressure release valve 172 may be in fluid communication with the expiratory conduit 107 between the expiratory proportional valve 116 and the expiratory sensor arrangement 182, as an example. The emergency expiratory pressure release valve 172 may be a magnetic pressure relief valve. The emergency expiratory pressure release valve 172 may be configured to open after achieving a predetermined pressure within the expiratory conduit 107. The emergency expiratory pressure release valve 172 may be configured to be biased toward a closed position when the pressure in the expiratory conduit 107 is below the predetermined pressure. However, when the predetermined pressure is reached and/or exceeded, the emergency expiratory pressure release valve 172 may be configured to move to an open position, allowing gas within the expiratory conduit 107 to escape and thus decreasing the pressure within the expiratory conduit 107.

In some non-limiting embodiments or aspects, one or more High Efficiency Particulate Air (HEPA) filters may be installed before and/or after each of the inlet and outlet positions of the manifold 170.

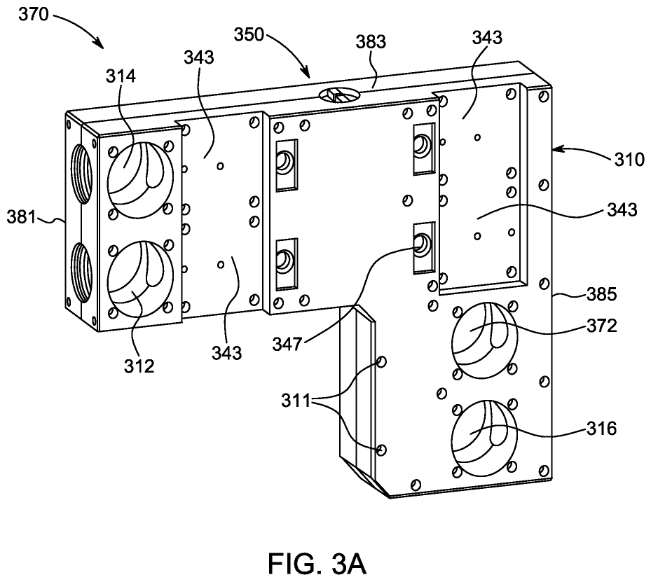

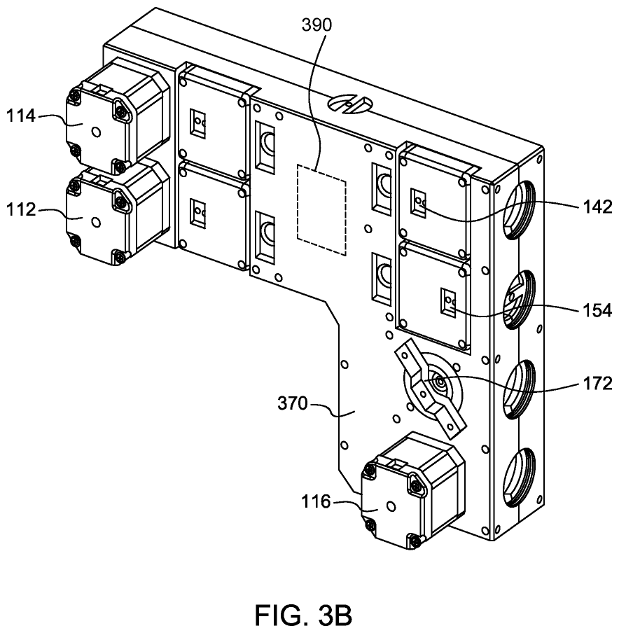

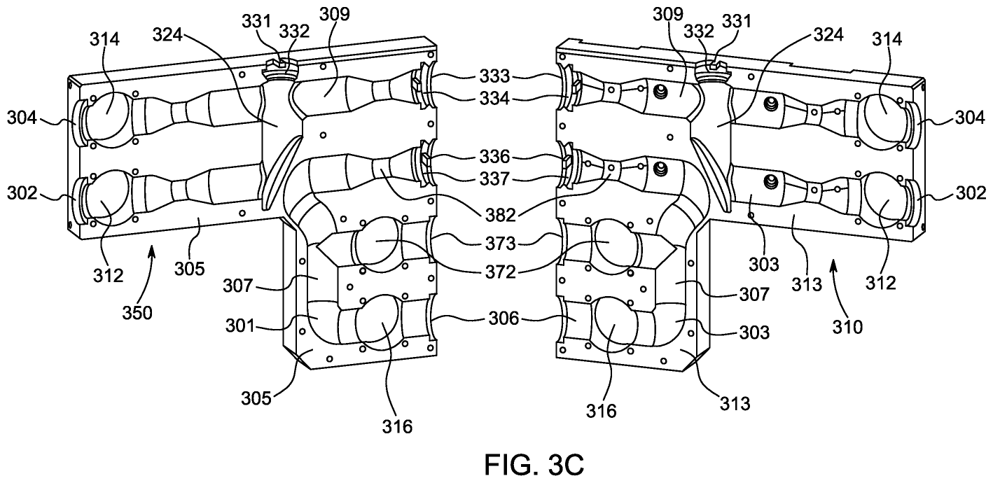

Referring now to FIGS. 3A-3C, shown is an example manifold 370 for a ventilator according to some non-limiting embodiments or aspects. The manifold 370 may be the same or similar to the manifold 170 shown in FIG. 1. The manifold 370 may be 3D printed or created from a mold. The manifold 370 may be made of a plastic material, although it will be appreciated that other materials may be used.

With continued reference to FIG. 3A, the manifold 370 may be made of two halves, a top manifold half 310 (e.g., a top plate) and a bottom manifold half 350 (e.g., a bottom plate). The top manifold half 310 may be connected to the bottom manifold half 350 in order to form the full manifold 370. The top manifold half 310 may be connected to the bottom manifold half 350 by inserting screws through screw holes 311 extending from the top manifold half 310 into the bottom manifold half 350. It will be appreciated that other fastening mechanisms may be used in addition to or alternative to screws. The manifold 370 may form an L shape.

With continued reference to FIG. 3C, in non-limiting embodiments or aspects, when the top manifold half 310 is connected to the bottom manifold half 350, it may make two distinct sets of conduits for the transportation of a gas. The surface of the conduits may be formed by indented surfaces of a bottom manifold half 301 and indented surfaces of a top manifold half 303. The raised surface of a bottom manifold half 305 and the raised surface of a top manifold half 313 come in contact when the top manifold half 310 is connected to the bottom manifold half 350. When the two halves are connected, the raised surface of the bottom manifold half 305 and the raised surface of the top manifold half 313 may create an air-tight connection such that air does not escape the conduits or at least air escape is minimal.

The first set of conduits may be an inspiratory conduit 309. The inspiratory conduit 309 may be the same or similar to the oxygen conduit 103, air conduit 105, and inspiratory conduit 109 of FIG. 1. The inspiratory conduit 309 may include at least one inlet. The inlets may include an oxygen inlet 302, an air inlet 304, and/or an emergency air inlet 331. The oxygen inlet 302 may be in fluid communication with an oxygen source, such as the oxygen source 102 of FIG. 1, by a tube, pipe, or other means of transporting oxygen. The air inlet 304 may be in fluid communication with an air source, such as the air source 104 of FIG. 1, by a tube, pipe, or other means of transporting air. The emergency air inlet 331 may be open to the atmosphere, or in fluid communication with another air source. The inspiratory conduit 309 may have at least one outlet, including a breathing apparatus outlet 333. The breathing apparatus outlet 333 may be in fluid communication with a breathing apparatus, such as the breathing apparatus 160 in FIG. 1, for use by a patient. The breathing apparatus outlet 333 may supply air for inhalation to a patient through the breathing apparatus. The breathing apparatus outlet 333 may be in fluid communication with the breathing apparatus by a tube, pipe, or other means for transporting air.

With continued reference to FIG. 3C, in non-limiting embodiments or aspects, the inspiratory conduit 309 from the air inlet 304 and the oxygen inlet 302 may combine in a chamber 324. The chamber 324 may be configured for mixing the gas flows from the oxygen inlet 302 and the air inlet 304. The chamber 324 may be the same or similar to the gas blender 124 of FIG. 1. In some non-limiting embodiments or aspects, the emergency air inlet 331 may direct air into the chamber 324, which may also be mixed with the gases from the oxygen inlet 302 and/or the air inlet 304. After mixing in the chamber 324, the combined gas may flow toward the breathing apparatus outlet 333.

With continued reference to FIGS. 3A-3C, in non-limiting embodiments or aspects, the air inlet 304 and oxygen inlet 302 may be on the same side of the manifold 370, such as the left side of a manifold 381. The emergency air inlet 331 may be located on a different side of the manifold 370 than the air inlet 304 and oxygen inlet 302, such as an upper side of the manifold 383. The breathing apparatus outlet 333 may be located on the opposite side of the manifold 370 from the air inlet 304 and oxygen inlet 302 (e.g., the right side of a manifold 385).

With continued reference to FIG. 3C, in non-limiting embodiments or aspects, the second distinct set of conduits may include an expiratory conduit 307. The expiratory conduit 307 may be independent of the inspiratory conduit 309. The expiratory conduit 307 may have at least one inlet and at least two outlets. The inlet may include a breathing apparatus inlet 337. The breathing apparatus inlet 337 may receive air from a breathing apparatus such as the breathing apparatus 160 of FIG. 1. The breathing apparatus inlet 337 may be in fluid communication with the breathing apparatus through a tube, pipe, or other means of transporting air. The breathing apparatus inlet 337 may receive air exhaled from a patient utilizing the breathing apparatus. Outlets for the expiratory conduit set may include an air sink outlet 306 and an atmosphere outlet 373. The air sink outlet 306 may be in fluid communication with a container, such as a tank, and may be the same or similar to the air sink 106 of FIG. 1. The atmosphere outlet 373 may allow air to be released into the atmosphere. In non-limiting embodiments or aspects, a HEPA filter may be incorporated into the manifold 370 or outside of the manifold 370 between the outlets and the atmosphere or air sink such that the exhaled air is filtered by the HEPA filter prior to entering the air sink or atmosphere.

With continued reference to FIGS. 3A-3C, in non-limiting embodiments or aspects, the breathing apparatus inlet 337, sink outlet 306, and atmosphere outlet 373 may all be located on the same side of the manifold 370 (e.g., the right side of the manifold 385).

With continued reference to FIGS. 3A-3C, the manifold 370 may include various spaces configured to receive valves, sensors, gas blenders, and/or other components. The manifold 370 may include an oxygen valve opening 312 for the insertion of the oxygen proportional valve 112, an air valve opening 314 for the insertion of the inspiratory proportional valve 114, an expiratory valve opening 316 for the insertion of an expiratory proportional valve 116, and a relief valve opening 372 for the insertion of an emergency expiratory pressure release valve 172. The proportional valves may be inserted such that the stepper motor may remain above the surface of the manifold. The stepper motor may be secured to the manifold 370 through the use of screws and/or other fastening mechanisms. The stepper motor may create an air tight-seal between the valve body and the manifold 370. The emergency expiratory pressure release valve 172 may be secured to the manifold 370 such as through the use of screws and/or other fastening mechanisms. In non-limiting embodiments, the oxygen valve opening 312, air valve opening 314, expiratory valve opening 316, and relief valve opening 372 may include a hole that extends through the top manifold half 310 such that at least a portion of a valve inserted in the openings may extend through the top manifold 310. In non-limiting embodiments, the oxygen valve opening 312, air valve opening 314, expiratory valve opening 316, and relief valve opening 372 may include a bottom surface on the bottom manifold half 350 such that a valve inserted into the openings through the top manifold half 310 cannot extend through the bottom surface of the bottom manifold half 310.

With continued reference to FIGS. 3A-3C, in non-limiting embodiments or aspects, the manifold 370 may include indented surfaces 343 for placement of flow sensors on the exterior of the top manifold half 310. The indented surfaces may be located above an orifice 382 that changes the cross-sectional area of the conduits 307, 309. The flow sensors may be secured to the manifold 370 through a fastening mechanism such as screws, adhesive, and/or the like.

With continued reference to FIGS. 3A-3C, in non-limiting embodiments or aspects, the manifold 370 may include at least one location configured for the insertion of a check valve 332, 334, 336. Check valves similar to or the same as the emergency breathing check valve 132, inspiratory check valve 134, and expiratory check valve 136 from FIG. 1 may be installed into the slots configured for the check valves 332, 334, 336.

With continued reference to FIGS. 3A-3C, in non-limiting embodiments or aspects, the manifold 370 may include at least one insertion location 347 configured for the insertion of pressure, humidity, and/or temperature sensors. The pressure, humidity, and/or temperature sensors insertion locations 347 may be located above a portion of the conduits 307, 309. A hole through the top manifold half 310 may allow instruments of the sensors to access the airflow within the conduits 307, 309.

With continued reference to FIG. 3B, in non-limiting embodiments or aspects, the manifold 370 with all components installed is modular in design. If a single component fails or has a malfunction, such as a valve, blender, or sensor, then the component can be replaced without significant impact to the remaining components or manifold. A computing device 390, such as a processor, may be included in the manifold to receive signals from the sensors and valves and to communicate the signals or the results of an analysis of the signals to a remote computing device. The computing device 390 may receive instructions from a remote computing device and/or determine instructions using predetermined sets of instructions and/or one or more artificial intelligence algorithms in order to operate the valves included in the manifold 370.

With continued reference to FIG. 3B, in non-limiting embodiments or aspects, the computing device 390 and sensors may allow control over the ventilator to control inspiratory pressure tidal volume positive-end expiratory pressure, respiratory rate, and inspiration to expiration ratio. The flow-sensor may be configured to sense the initiation of a breath from the patient (e.g., by sensing sudden decreases in flow and pressure indicating that the patient initiated a breath). In the event that the manifold 370 itself fails, all of the components (e.g., valves, blender, and sensors) can be moved to a new manifold 370. This allows for simpler repairs of the manifold 370 compared to existing ventilator systems. Repairs can be completed quicker allowing the ventilator to return to operation with less downtime. Due to the ease of production of the individual parts, spare parts can either be kept on hand or obtained quickly compared to ventilator components typically found in the medical supply chain.

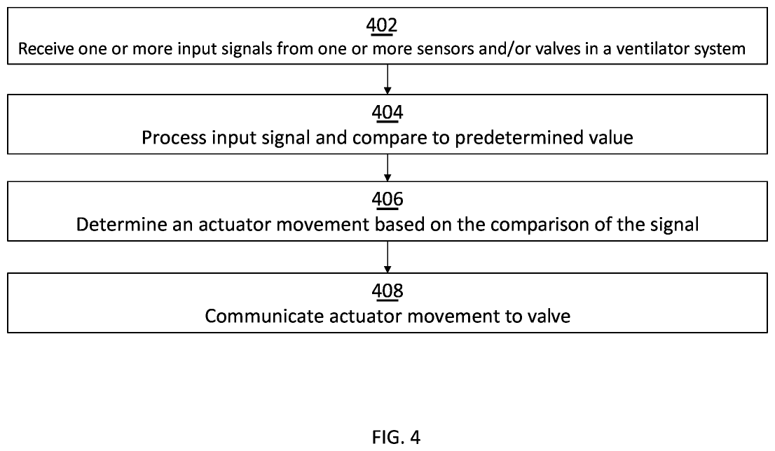

Referring now to FIG. 4, shown is a method of operating a ventilator according to some non-limiting embodiments or aspects. The ventilator may be controlled by a central controller. The central controller may control more than one ventilator simultaneously. The central controller may include at least one computing device, such as a processor, and at least one printed circuit board (PCB) arranged in communication with a plurality of ventilators. The central controller may be arranged local to the ventilators, remote from the ventilators but in the same facility, remote from the ventilators and facility, and/or the like. The central controller may interface with the inspiratory sensor arrangement 180 and expiratory sensor arrangement 182 of FIG. 1, as well as the actuator 204 of FIG. 2. In Step 402, the processor of the central controller may receive one or more input signals from one or more sensors and/or valves in the ventilator system. The input signal may include a pressure, temperature, flow rate, humidity, air quality, and/or the like of a point in a conduit within the ventilator system. The input signal may also include a motorized proportional valve position, such as the closed/open condition of the valve or the position of the valve actuator.

With continued reference to FIG. 4, in step 404, the central controller may process the input signal. Processing the input signal may include comparing the input signal to a comparison value. The comparison value may be based on a machine learning algorithm or a predetermined value. The comparison value may be based on input signals received from other sensors associated with the same ventilator. For example, a comparison value for a flow rate may depend on the current pressure and air quality of the air in the ventilator. The central controller may determine if the flow rate is higher or lower than the comparison value. The comparison value may represent an ideal value for the signal based on one or more conditions of the ventilator. In examples in which the comparison value is determined with a machine learning algorithm, the machine learning algorithm may be based on the operating history of several ventilators in a single system or across multiple systems such that it learns an optimal comparison value based on prior results and operating conditions.

With continued reference to FIG. 4, in step 406 the central controller may determine an actuator movement. The actuator movement may be based on the input signal, such as the comparison of the signal to the comparison value. For example, if the central controller determines that the flow rate should be increased based on the comparison to the comparison value, then the central controller may determine that the actuator should be moved to increase the flow rate. The actuator movement may be determined based on the input signal based on the machine learning algorithm. The actuator movement may be a movement that results in the motorized proportional valve actuator moving the valve toward a closed position or an open position.

With continued reference to FIG, 4, in step 408, the central controller may communicate an actuator movement command based on the determined actuator movement to the motorized proportional valve actuator associated with the ventilator. The central controller may communicate the actuator movement command through wireless communication (e.g., Bluetooth® and/or Wi-Fi®).

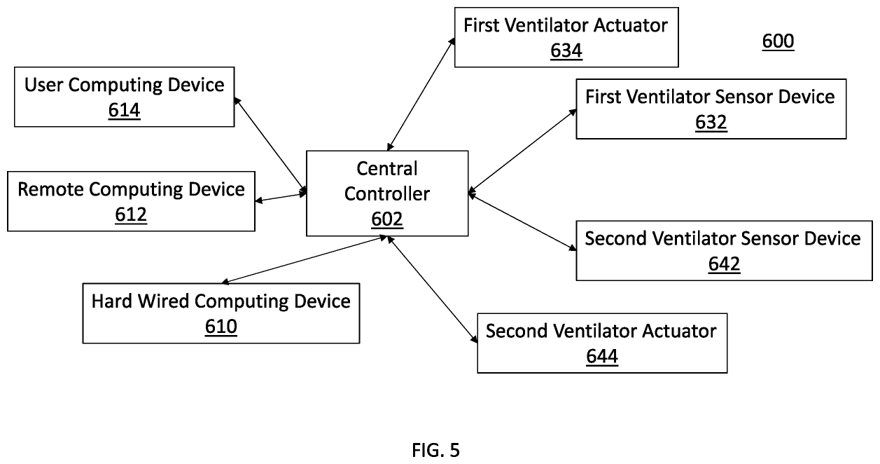

Referring now to FIG. 5, shown is a remote control system 600 for operating multiple ventilators according to non-limiting embodiments. A central controller 602 may include a computing device configured to receive inputs from multiple ventilators. The central controller 602 may be built on a circuit board that is small in size (e.g., smaller than a postage stamp). The central controller 602 may be configured to complete real-time tasks. The central controller 602 may be arranged local to one or more ventilators, remote from the one or more ventilators but in the same facility, remote from the one or more ventilators and facility, and/or the like and may communicate with the sensors and/or motorized proportional valve actuators associated with the one or more ventilators (e.g., at least two ventilators) through wireless communication. For example, it may receive a signal from a sensor device associated with a first ventilator (e.g., a first ventilator sensor device 632) and receive a second signal from a sensor device associated with a second ventilator (e.g., a second ventilator sensor device 642). The signals may be associated with pressure, flow rate, temperature, air quality, and/or humidity associated with pressurized air within a conduit of the ventilator.

With continued reference to FIG. 5, in non-limiting embodiments or aspects, if the central controller 602 receives an input signal from the first ventilator sensor device 632 and/or second ventilator sensor device 642 that represents an unsafe condition or the input signal is used to determine that an unsafe condition exists, such as an unsafe pressure, tidal volumes, or minute ventilation, the central controller may initiate one or more alarms. The alarms may be visible and/or audible, and may be displayed on and/or output by a user computing device 614 or any other computing device in communication with the central controller. The alarms may be a component of the manifold (e.g., devices for making auditory noise and/or visual lights configured to initiate when an unsafe condition exists and may be fastened to the manifold). A condition may be determined to be unsafe if the input signal satisfies a predetermined threshold.

With continued reference to FIG. 5, in non-limiting embodiments or aspects, the central controller 602 may communicate, either wirelessly or wired, the signals, numerical representation of the signals, and/or a graphical representation of the signals to a remote computing device 612, the user computing device 614, and/or a hardwired computing device 610. The hardwired computing device 610 may be hard wired to the ventilator. A user may visualize the signals on a graphical user interface (UI) on a display device associated with the user computing device 614, remote computing device 612, and/or hardwired computing device 610.

With continued reference to FIG. 5, in non-limiting embodiments or aspects, a graphical user interface (GUI) may be displayed through a mobile application on the remote computing device 612, the user computing device 614, and/or the hardwired computing device 610. The GUI may display patient ventilator conditions (e.g., pressure, flow, volume tracings, and/or the like) and user adjusted settings. The GUI may display the patient ventilator conditions for multiple patients simultaneously on the GUI. The display of multiple patient properties simultaneously may allow for more direct monitoring of each patient during high patient caseload events or during workforce reduction. The user adjusted settings may be used to adjust the ventilators for multiple patients simultaneously. Personal identifiable information may be protected by encryption (e.g., using a FIPS 140-2 protocol).

In some non-limiting embodiments or aspects, the central controller 602 may communicate suggested setting adjustments to the user through the GUI. The suggested setting adjustments may be determined based on a machine learning algorithm and input signals from the air flow and pressure of the ventilator. For example, the machine learning algorithm may determine the suggested setting adjustments based on previously collected data, including data from expert and novice ventilator operators, which may be stored in a storage system, such as a remote (e.g., cloud-based) data storage system. The storage system may be configured for extensive waveform data storage. In non-limiting embodiments, expert and novice data is used to train the machine learning training to ensure more reliable performance as never-before-seen may be generalized more easily. The initial set of expert and novice data may be based on lung simulator operations, for example. The simulator operations may be completed while a manifold is attached to a simulator. The machine learning algorithm may identify and automatically correct unsatisfactory conditions, such as those leading to dynamic hyperinflation that may further injure the lungs. The machine learning algorithm may enable less experienced operators who are not trained extensively in critical care to provide improved ventilation when more trained personnel are not available.

With continued reference to FIG. 5, in non-limiting embodiments or aspects, in response to the visualization of the signals, the user may input an actuator movement command for one or more actuators associated with the signals. The actuator movement command may be communicated to the central controller 602 from the remote computing device 612, the user computing device 614, and/or the hardwired computing device 610. In some non-limiting embodiments or aspects, the actuator movement command may be determined by the machine learning algorithm and may be communicated automatically or at the user's command. The actuator movement command may direct the movement of the actuator that results in the actuator moving toward the open or closed position

With continued reference to FIG. 5, in non-limiting embodiments or aspects, in response to receiving the actuator movement command, the central controller 602 may communicate the actuator movement command to the selected one or more actuators, such as a first ventilator actuator 634 and/or a second ventilator actuator 644. In response to receiving the actuator movement command, the first ventilator actuator 634 and/or the second ventilator actuator 644 may operate the actuator based on the actuator movement command. The movement of the actuator may result in the first ventilator actuator 634 and/or the second ventilator actuator 644 moving toward the open or closed position.

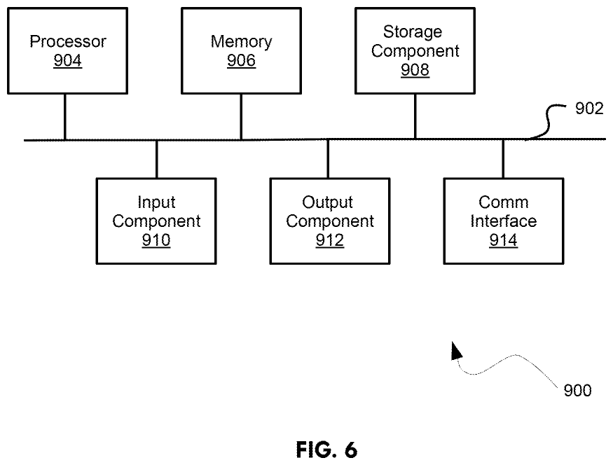

Referring now to FIG. 6, shown is a diagram of example components of a computing device 900 for implementing and performing the systems and methods described herein according to non-limiting embodiments. The computing device 900 may be the same or similar to the user computing device 614, remote computing device 612, hardwired computing device 610, and/or central controller 602 of FIG. 5. In some non-limiting embodiments, device 900 may include additional components, fewer components, different components, or differently arranged components than those shown in FIG. 6. Device 900 may include a bus 902, a processor 904, memory 906, a storage component 908, an input component 910, an output component 912, and a communication interface 914. The bus 902 may include a component that permits communication among the components of the device 900. In some non-limiting embodiments, the processor 904 may be implemented in hardware, firmware, or a combination of hardware and software. For example, the processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by the processor 904.

With continued reference to FIG. 6, the storage component 908 may store information and/or software related to the operation and use of the device 900. For example, the storage component 908 may include a hard disk (e.g., a magnetic disk, an optical disk, a magneto-optic disk, a solid state disk, etc.) and/or another type of computer-readable medium. The input component 910 may include a component that permits the device 900 to receive information, such as via user input (e.g., a touch screen display, a keyboard, a keypad, a mouse, a button, a switch, a microphone, etc.). Additionally, or alternatively, the input component 910 may include a sensor for sensing information (e.g., a photo-sensor, a thermal sensor, an electromagnetic field sensor, a global positioning system (GPS) component, an accelerometer, a gyroscope, an actuator, etc.). The output component 912 may include a component that provides output information from the device 900 (e.g., a display, a speaker, one or more light-emitting diodes (LEDs), etc.). The communication interface 914 may include a transceiver-like component (e.g., a transceiver, a separate receiver and transmitter, etc.) that enables device 900 to communicate with other devices, such as via a wired connection, a wireless connection, or a combination of wired and wireless connections. The communication interface 914 may permit the device 900 to receive information from another device and/or provide information to another device. For example, the communication interface 914 may include an Ethernet interface, an optical interface, a coaxial interface, an infrared interface, a radio frequency (RF) interface, a universal serial bus (USB) interface, a Wi-Fi® interface, a cellular network interface, and/or the like.

The device 900 may perform one or more processes described herein. Device 900 may perform these processes based on the processor 904 executing software instructions stored by a computer-readable medium, such as the memory 906 and/or the storage component 908. A computer-readable medium may include any non-transitory memory device. A memory device includes memory space located inside of a single physical storage device or memory space spread across multiple physical storage devices. Software instructions may be read into memory 906 and/or storage component 908 from another computer-readable medium or from another device via the communication interface 914. When executed, software instructions stored in the memory 906 and/or the storage component 908 may cause processor 904 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

Although embodiments have been described in detail for the purpose of illustration, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A ventilator comprising:
at least one motorized proportional valve actuator, each motorized proportional valve actuator comprising a stepper motor and an actuator, the actuator connected to the stepper motor and configured to output pressurized air by controlling a pressure on a valve diaphragm;
a conduit providing for fluid communication of the pressurized air to a breathing apparatus; and
a sensor arrangement in fluid communication with the conduit between the at least one motorized proportional valve actuator and the breathing apparatus, the sensor arrangement comprising: (i) an intake manifold configured to output a restricted flow of air from the pressurized air transported in the conduit, and (ii) a sensor device in fluid communication with an outlet of the intake manifold, the sensor device configured to measure an air pressure of the conduit based on the restricted flow of air.

2. The ventilator of claim 1, further comprising a processor in communication with the sensor device, the processor configured to determine a temperature compensation and a humidity compensation.

3. The ventilator of claim 1, wherein the sensor device is configured to measure a pressure range of −200 cm H₂O to 200 cm H₂O and a flow rate range of 0 to 100 L/min.

4. The ventilator of claim 1, wherein the motorized proportional valve actuator comprises a plurality of motorized proportional valve actuators comprising an oxygen proportional valve configured to adjust oxygen concentration levels, an inspiratory proportional valve configured to adjust air pressure of inspiratory air, and an expiratory proportional valve configured to adjust air pressure of expiratory air.

5. The ventilator of claim 4, wherein the inspiratory proportional valve actuator is configured to operate in an inspiratory range of 0-120 cm H₂O and the expiratory pressure motorized proportional valve actuator is configured to operate in an expiratory range of 0-25 cm H₂O.

6. The ventilator of claim 1, wherein the intake manifold comprises a top plate and a bottom plate, wherein connecting the top plate to the bottom plate forms an inspiratory conduit and an expiratory conduit, the inspiratory conduit being independent of the expiratory conduit.

7. A central controller for a ventilator comprising:
one or more processors programmed and/or configured to:
receive an input signal from a sensor arrangement in fluid communication with a conduit between at least one motorized proportional valve actuator and a breathing apparatus providing for fluid communication of pressurized air to the breathing apparatus, the sensor arrangement comprising: (i) an intake manifold configured to output a restricted flow of air from the pressurized air transported in the conduit, and (ii) a sensor device in fluid communication with an outlet of an intake manifold, the sensor device configured to measure an air pressure of the conduit based on the restricted flow of air;
determine an actuator movement based on the input signal; and
communicate an instruction based on the actuator movement to a motorized proportional valve actuator comprising a stepper motor and an actuator, the actuator connected to the stepper motor and configured to output the pressurized air by controlling a pressure on a valve diaphragm, wherein the instruction directs the stepper motor to complete the actuator movement to change the pressure on the valve diaphragm.

8. The central controller of claim 7, wherein the central controller is wireless and arranged remote from the ventilator, and configured to be in wireless communication with at least one motorized proportional valve actuator of at least two ventilators.

9. The central controller of claim 7, wherein the central controller is in communication with a user device, wherein the central controller operates the at least one motorized proportional valve actuator based on an input signal received from the user device.

10. The central controller of claim 7, wherein determining an actuator movement based on the input signal is determined based on a machine learning algorithm.

11. A method of operating a ventilator, comprising:
receiving, with at least one processor, an input signal from at least one sensor arrangement in fluid communication with a conduit, the conduit establishing fluid communication between at least one motorized proportional valve actuator and a breathing apparatus, the sensor arrangement comprising: (i) an intake manifold configured to output a restricted flow of air from pressurized air transported in the conduit, and (ii) at least one sensor device in fluid communication with an outlet of the intake manifold, the sensor device configured to measure an air pressure of the conduit based on the restricted flow of air; and
adjusting, with at least one processor, a stepper motor of a motorized proportional valve actuator based on comparing the input signal to a predetermined value, wherein the motorized proportional valve actuator comprises an actuator connected to the stepper motor, the actuator configured to output pressurized air by controlling a pressure on a valve diaphragm.

12. The method of claim 11, further comprising determining, with at least one processor, a temperature compensation and a humidity compensation of the at least one sensor device.

13. The method of claim 11, wherein the sensor device is configured to measure a pressure range of −200 cm H₂O to 200 cm H₂O and a flow rate range of 0 to 100 L/min

14. The method of claim 11, wherein the motorized proportional valve actuator comprises a plurality of motorized proportional valve actuators including an oxygen proportional valve configured to adjust oxygen concentration levels, an inspiratory proportional valve configured to adjust air pressure of inspiratory air, and an expiratory proportional valve configured to adjust air pressure of expiratory air.

15. The method of claim 14, wherein the inspiratory pressure motorized proportional valve actuator is configured to operate in an inspiratory range of 0-120 cm H₂O and the expiratory pressure motorized proportional valve actuator is configured to operate in an expiratory range of 0-25 cm H₂O.

16. The method of claim 11, wherein the intake manifold comprises a top plate and a bottom plate, wherein connecting the top plate to the bottom plate forms an inspiratory conduit and an expiratory conduit, the inspiratory conduit being independent of the expiratory conduit.
